(19) United States
(12) Patent Application Publication
(10) Pub. No.: US 2018/0146890 A1
(43) Pub. Date: May 31, 2018

(54) APPARATUS AND METHOD FOR RECOGNIZING GAIT STATE

(71) Applicant: Samsung Electronics Co., Ltd., Suwon-si (KR)

(72) Inventors: Kyung-Rock KIM, Yongin-si (KR); Joon-Kee CHO, Yongin-si (KR); Youngbo SHIM, Seoul (KR); Bokman LIM, Yongin-si (KR); Taesin HA, Seongnam-si (KR)

(73) Assignee: Samsung Electronics Co., Ltd., Suwon-si (KR)

(21) Appl. No.: 15/489,126

(22) Filed: Apr. 17, 2017

(30) Foreign Application Priority Data
Nov. 25, 2016 (KR) 10-2016-0158233

(51) Int. Cl. A61B 5/11; A61B 5/00; A61H 3/00

(52) U.S. Cl. CPC A61B 5/112; A61B 5/1121; A61B 5/7264; A61B 2562/0219; A61B 5/6823; A61B 5/6828; A61H 3/00; A61B 5/1126

(57) ABSTRACT

A gait state recognizing apparatus may include an inertial measurement unit (IMU) sensor configured to measure a movement of at least one leg of a user and a processor configured to calculate a rotation angle and an angular velocity of the at least one leg based on the measured movement of the at least one leg, and the processor may be configured to calculate the rotation angle of the at least one leg relative to a direction of gravity from the movement of the at least one leg and the angular velocity of the at least one leg relative to the direction of gravity based on a trend of the rotation angle.

### CROSS-REFERENCE TO RELATED APPLICATION

This application claims priority under 35 U.S.C. § 119 to Korean Patent Application No. 10-2016-0158233, filed on Nov. 25, 2016, in the Korean Intellectual Property Office, the entire contents of which are incorporated herein by reference.

### BACKGROUND

### 1. Field

At least one example embodiment relates to an apparatus and/or method for recognizing a gait state. For example, at least some one example embodiment relates to an apparatus and/or method for recognizing a gait state during a gait cycle of a user using an inertial measurement unit (IMU).

### 2. Description of the Related Art

When a user using a movement assistance apparatus is provided general movement assistance without possible user actions having been classified, the movement assistance may not be effective. For example, when the user is provided general movement assistance without possible user actions having been classified, the movement assistance may not be based on a feature of the particular user action.

For a user using a movement assistance apparatus, it is desirable for the apparatus to provide movement assistance appropriate for each action the user performs. That is, for the user it is desirable for the apparatus to provide customized (or, alternatively, optimized) movement assistance by not providing unnecessary movement assistance.

### SUMMARY

Some example embodiments relate to a gait state recognizing apparatus.

In some example embodiments, the gait state recognizing apparatus may include at least one inertial measurement unit (IMU) sensor configured to measure a movement of at least one leg of a user; and a processor configured to, calculate a rotation angle and an angular velocity of the at least one leg based on the measured movement of the at least one leg, and determine a transition between gait states of the user based on one or more of the rotation angle and the angular velocity of the at least one leg.

In some example embodiments, the at least one leg includes a left leg of the user and a right leg of the user, and the processor is configured to, determine the transition between the gait states based on one or more of (i) the rotation angle of the right leg crossing the rotation angle of the left leg and (ii) the angular velocity of the right leg crossing the angular velocity of the left leg, calculate the rotation angle by calculating the rotation angle of the at least one leg relative to a direction of gravity from the movement of the at least one leg, and calculate the angular velocity by calculating angular velocity of the at least one leg relative to the direction of gravity based on a trend of the rotation angle.

In some example embodiments, the processor is configured to determine whether the user is walking based on the angular velocity of the at least one leg.

In some example embodiments, the processor is configured to determine the gait states of the user based on a trend of the rotation angle and a trend of the angular velocity of the at least one leg.

In some example embodiments, the processor is configured to determine the gait states using a finite state machine (FSM), the FSM including at least one state associated with a gait cycle of the user.

In some example embodiments, the at least one leg includes a left leg of the user and a right leg of the user, and the gait states include a state in which the left leg swings, a state in which the left leg lands, a state in which the right leg swings, and a state in which the right leg lands.

In some example embodiments, the at least one leg includes a left leg of the user and a right leg of the user, and the at least one IMU sensor comprises: a first IMU sensor configured to measure a movement of the right leg, and a second IMU configured to measure a movement of the left leg.

In some example embodiments, the at least one IMU sensor further comprises: a third IMU sensor configured to measure a movement of an upper body of the user, and, wherein the processor is configured to determine the gait states based on the movement of the upper body.

In some example embodiments, one IMU sensor included in the at least IMU sensor is configured to measure movement associated with a portion of the user such that a relative movement of the portion with respect to the at least one leg is less than relative movement with respect to a joint of the at least one leg.

In some example embodiments, one IMU sensor included in the at least one IMU sensor is configured to measure movement of a thigh of the user.

Other example embodiments relate to a method of recognizing a gait state.

In some example embodiments, the method may include measuring, via at least one inertial measurement unit (IMU) sensor, a movement of at least one leg of a user; calculating a rotation angle and an angular velocity of the at least one leg based on the movement of the at least one leg; determining whether the user is walking based on the angular velocity of the at least one leg; and determining a transition between gait states of the user based on one or more of the rotation angle and the angular velocity of the at least one leg.

In some example embodiments, the at least one leg includes a left leg of the user and a right leg of the user, the determining determines the transition between the gait states based on one or more of (i) the rotation angle of the right leg crossing the rotation angle of the left leg and (ii) the angular velocity of the right leg crossing the angular velocity of the left leg, and the calculating the rotation angle includes calculating the rotation angle of the at least one leg relative to a direction of gravity from the movement of the at least one leg, and the calculating the angular velocity includes calculating the angular velocity of the at least one leg relative to the direction of gravity based on a trend of the rotation angle of the at least one leg.

In some example embodiments, the method further includes determining the gait states of the user based on a trend of the rotation angle of the at least one leg and a trend of the angular velocity of the at least one leg when the user is walking.

In some example embodiments, the determining the gait states determines the gait states based on a finite state machine (FSM), the FSM including at least one state associated with a gait cycle of the user.

In some example embodiments, the at least one leg includes a left leg of the user and a right leg of the user, and at least one IMU sensor includes, a first IMU sensor configured to measure a movement of the right leg and a second IMU sensor configured to measure a movement of the left leg.

Some example embodiments relate to a non-transitory computer-readable storage medium storing a program that, when executed by a processor, configures the processor to perform the method of recognizing the gate state.

Additional aspects of example embodiments will be set forth in part in the description which follows and, in part, will be apparent from the description, or may be learned by practice of the disclosure.

### BRIEF DESCRIPTION OF THE DRAWINGS

These and/or other aspects will become apparent and more readily appreciated from the following description of example embodiments, taken in conjunction with the accompanying drawings of which:

FIG. 1 is a block diagram illustrating a gait state recognizing apparatus according to at least one example embodiment;

FIG. 2 is a block diagram illustrating a gait state recognizing apparatus according to at least one example embodiment;

FIG. 3 illustrates an example of an implementation of a gait state recognizing apparatus according to at least one example embodiment;

FIG. 4 illustrates an example of an implementation of a gait state recognizing apparatus according to at least one example embodiment;

FIG. 5 illustrates an example of an implementation of a gait state recognizing apparatus according to at least one example embodiment;

FIG. 6 illustrates an operation principle of a gait state recognizing apparatus according to at least one example embodiment;

FIG. 7 is a graph illustrating an experimental result of a gait state recognizing apparatus according to at least one example embodiment;

FIG. 8 is a graph illustrating a process of recognizing gait states during a gait cycle of a user using a gait state recognizing apparatus according to at least one example embodiment;

FIG. 9 illustrates a finite state machine (FSM) used to recognize a movement of a user by a gait state recognizing apparatus according to at least one example embodiment; and

FIG. 10 is a flowchart illustrating a method of recognizing a gait state according to at least one example embodiment.

### DETAILED DESCRIPTION

Hereinafter, some example embodiments will be described in detail with reference to the accompanying drawings. Regarding the reference numerals assigned to the elements in the drawings, it should be noted that the same elements will be designated by the same reference numerals, wherever possible, even though they are shown in different drawings. Also, in the description of example embodiments, detailed description of well-known related structures or functions will be omitted when it is deemed that such description will cause ambiguous interpretation of the present disclosure.

It should be understood, however, that there is no intent to limit this disclosure to the particular example embodiments disclosed. On the contrary, example embodiments are to cover all modifications, equivalents, and alternatives falling within the scope of the example embodiments. Like numbers refer to like elements throughout the description of the figures.

In addition, terms such as first, second, A, B, (a), (b), and the like may be used herein to describe components. Each of these terminologies is not used to define an essence, order or sequence of a corresponding component but used merely to distinguish the corresponding component from other component(s). It should be noted that if it is described in the specification that one component is “connected”, “coupled”, or “joined” to another component, a third component may be “connected”, “coupled”, and “joined” between the first and second components, although the first component may be directly connected, coupled or joined to the second component.

The terminology used herein is for the purpose of describing particular embodiments only and is not intended to be limiting. As used herein, the singular forms “a,” “an,” and “the,” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Various example embodiments will now be described more fully with reference to the accompanying drawings in which some example embodiments are shown. In the drawings, the thicknesses of layers and regions are exaggerated for clarity.

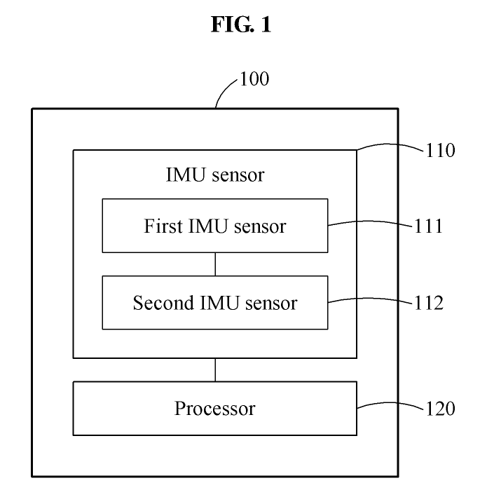

FIG. 1 is a block diagram illustrating a gait state recognizing apparatus 100 according to at least one example embodiment.

Referring to FIG. 1, in an example embodiment, the gait state recognizing apparatus 100 includes an inertial measurement unit (IMU) sensor 110 and a processor 120.

Generally, an encoder sensor configured to measure an angle of a joint, for example, a hip joint or a knee joint, is used to obtain information associated with a gait state. However, using the encoder sensor an accuracy of gait state recognition may be relatively low when a user makes a gesture that causes a relative movement between a body of the user and a gait state recognizing apparatus because the encoder sensor is disposed around a joint to measure a joint angle at a position where a movement of the body of the user has relatively great influence. Compared to the encoder sensor, the IMU sensor 110 of the gait state recognizing apparatus 100 may obtain the information associated with the gait state by measuring a movement of a leg relative to a direction of gravity through a combination of IMU sensors without the encoder sensor. Therefore, in some example embodiments, the gait state recognizing apparatus 100 may accurately recognize a gait state by using the IMU sensor 110 and, thus, the gait state recognizing apparatus 100 may not utilize an encoder sensor to recognize the gait state.

The IMU sensor 110 includes a first IMU sensor 111 and a second IMU sensor 112. For example, the first IMU sensor 111 may be disposed on a right leg to measure a movement of the right leg of the user, and the second IMU sensor 112 may be disposed on a left leg to measure a movement of the left leg of the user. For example, the first IMU sensor 111 and the second IMU sensor 112 may be provided to measure six degrees of freedom (6 DoF) movement of each leg.

The first IMU sensor 111 and the second IMU sensor 112 may be disposed on a portion where the shaking of the gait state recognizing apparatus 100 has little influence, that is, a portion where a relative movement between the gait state recognizing apparatus 100 and a body of a user is relatively small. For example, the relative movement between the gait state recognizing apparatus 100 and the body of the user may be relatively great around, for example, a hip joint, a knee joint, and an ankle joint, due to a rotation of the joint. Thus, unlike an encoder sensor, which may be installed at a joint, the first IMU sensor 111 and the second IMU sensor 112 may be disposed on a portion where the relative movement with respect to the body of the user is smallest. For example, the first IMU sensor 111 and the second IMU sensor 112 may be disposed one on each thigh of the user because a curvature of a surface of the thigh is relatively low and the rotation of the joint has little influence on the thigh.

The processor 120 may be any processing circuitry such as, but not limited to, a processor, Central Processing Unit (CPU), a controller, an arithmetic logic unit (ALU), a digital signal processor, a microcomputer, a field programmable gate array (FPGA), an Application Specific Integrated Circuit (ASIC), a System-on-Chip (SoC), a programmable logic unit, a microprocessor, or any other device capable of performing operations in a defined manner.

According to one or more example embodiments, the processing circuitry may be described as including various functional units that perform various operations and/or functions to increase the clarity of the description. However, in one or more example embodiments, the various operations and/or functions of the functional units may be performed by other ones of the functional units. Further, the processing circuitry may perform the operations and/or functions of the various functional units without sub-dividing the operations and/or functions into these various functional units.

The processor 120 may be included in a frame of the gait state recognizing apparatus 100 and provided in a personal computer (PC), for example, a desktop computer and a laptop computer, a piece of mobile user equipment, or a server via a wired or wireless connection. A function of the proposed processor 120 is not limited by a form in which it is implemented.

The processing circuitry may be configured, through a layout design and/or execution of computer readable instructions stored in a memory (not shown), as a special purpose computer to determine a gait state of the user based on data from the IMU sensor 110.

The memory may include a nonvolatile memory device, a volatile memory device, a non-transitory storage medium, or a combination of two or more of the above-mentioned devices. For example, the memory may include one or more of a Read Only Memory (ROM), Random Access Memory (RAM), Compact Disk-Read Only Memories (CD-ROMs), magnetic tapes, floppy disks, and an optical recording medium.

For example, the processor 120 may calculate a rotation angle of each leg relative to a direction of gravity based on a movement of each leg measured by the IMU sensor 110 and calculate an angular velocity of each leg relative to the direction of gravity based on a trend of the rotation angle of each leg relative to the direction of gravity as time elapses. A pivot of each of the rotation angle and the angular velocity may be set to be a desired (or, alternatively, a predetermined) initial value or customized to be a point associated with an actual position of a hip joint of the user.

The processor 120 may determine whether the user is walking based on the calculated angular velocity of each leg. For example, in response to an absolute value of the angular velocity being greater than a desired (or, alternatively, a predetermined) threshold value, the processor 120 determines that the user is walking. Through an operation of determining whether the user is walking, a gait state of the user may be recognized only when the user is walking.

The processor 120 may determine the gait state of the user based on a trend of the rotation angle and a trend of the angular velocity of each leg. For example, the processor 120 defines a plurality of gait states included in a gait cycle of the user and determines a gait state of the user among the plurality of gait states based on the trend of the rotation angle and the trend of the angular velocity of each leg.

The plurality of gait states included in the gait cycle of the user may be defined and recognized using a finite state machine (FSM). For example, in a case of a general gait pattern in which the user walks in a forward direction, the gait cycle of the user may include a state in which a left leg swings, a state in which the left leg lands, a state in which a right leg swings, and a state in which the right leg lands. Detailed description of a method of recognizing the gait state using the FSM is provided below.

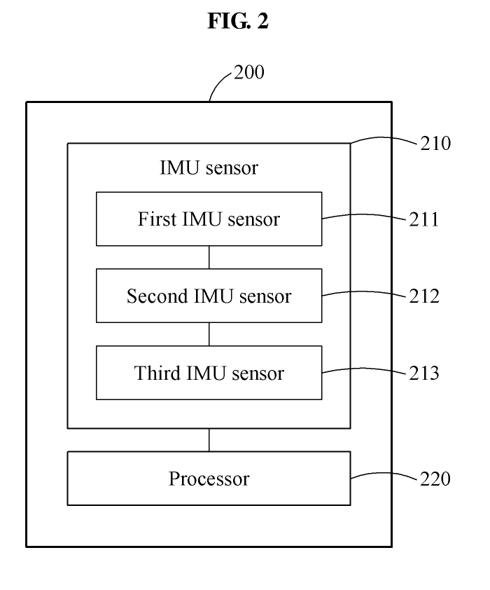

FIG. 2 is a block diagram illustrating a gait state recognizing apparatus 200 according to at least one example embodiment.

Referring to FIG. 2, the gait state recognizing apparatus 200 includes an inertial measurement unit (IMU) sensor 210 and a processor 220.

Similar to the gait state recognizing apparatus 100 of FIG. 1, the gait state recognizing apparatus 200 of FIG. 2 also includes the IMU sensor 210 configured to obtain information associated with a gait state based on a combination of IMU sensors without an encoder sensor.

The IMU sensor 210 includes a first IMU sensor 211, a second IMU sensor 212, and a third IMU sensor 213. For example, the first IMU sensor 211 is disposed on a right leg to measure a movement of the right leg of a user, the second IMU sensor 212 is disposed on a left leg to measure a movement of the left leg of the user, and the third IMU sensor 213 is disposed on a waist to measure a movement of an upper body of the user. For example, the first IMU sensor 211, the second IMU sensor 212, and the third IMU sensor 213 are provided to measure six degrees of freedom (6 DoF) movement of each leg and the upper body.

Because the gait state recognizing apparatus 200 further includes the third IMU sensor 213 configured to measure the movement of the upper body of the user, the movement of the upper body measured by the third IMU sensor 213 may be additionally used to determine whether the user is walking and/or to recognize the gait state of the user. By using such a method, an accuracy of determining whether the user is walking and/or recognizing the gait state may be enhanced.

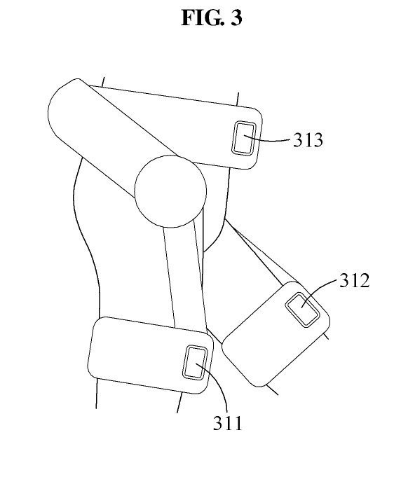

FIGS. 3 through 5 illustrate examples of implementations of a gait state recognizing apparatus according to at least one example embodiment. When a method of measuring a rotation angle of a hip joint or a knee joint using an encoder sensor is used, the encoder sensor may be disposed on a portion where a rotation of a joint has influence. However, referring to FIGS. 3 through 5, a sensor may be disposed on a waist, a thigh, or a calf where the rotation of the joint has relatively little influence. Thus, recognition errors caused by relative movements between a body of a user and a sensor as illustrated in FIGS. 3 through 5, may be reduced.

Referring to FIG. 3, FIG. 3 illustrates a first IMU sensor 311 fastened to a thigh of a right leg to measure a movement of the right leg of the user, a second IMU sensor 312 fastened to a thigh of a left leg to measure a movement of the left leg of the user, and a third IMU sensor 313 fastened to a waist to measure a movement of an upper body of the user.

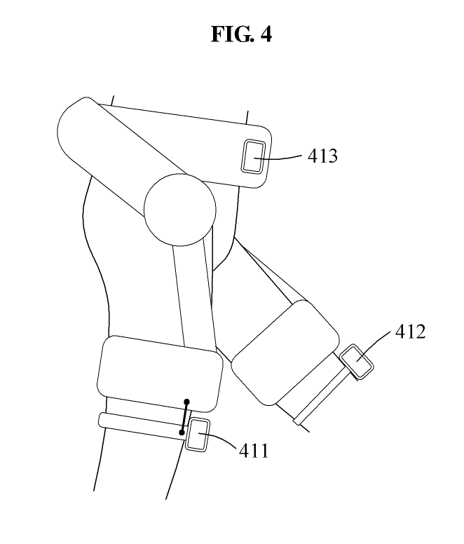

Referring to FIG. 4, FIG. 4 illustrates a first IMU sensor 411 to measure a movement of a right leg of the user, a second IMU sensor 412 to measure a movement of a left leg of the user, and a third IMU sensor 413 to measure a movement of an upper body of a user. The first IMU sensor 411 and the second IMU sensor 412 may include an additional device used to fasten a sensor to reduce recognition errors caused by shaking of a gait assistance apparatus or shaking of a gait state recognizing apparatus. For example, as illustrated in FIG. 4, the first IMU sensor 411 and the second IMU sensor 412 are fastened one on each thigh of the user using the additional device being separate from a frame of the gait assistance apparatus or the gait state recognizing apparatus. The additional devices may be adjustable straps, bands or belts configured to secure to the thighs of the user.

In this example, the gait state recognizing apparatus may further include a wired or wireless communication device to transmit a result of measuring the movements by the first IMU sensor 411 and the second IMU sensor 412 to, for example, the processor 220.

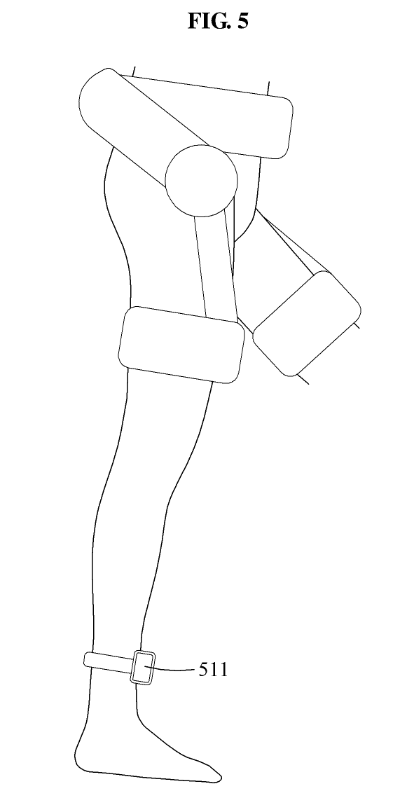

FIG. 5 illustrates an IMU sensor 511 fastened to a calf of a right leg to measure a movement of the right leg of a user. However, example embodiments are not limited thereto. For example, the IMU sensor 511 may be fastened to a calf of a left leg of the user. The IMU sensor 511 may be fastened to the calf of the user using an additional device being separate from a frame of a gait assistance apparatus or a gait state recognizing apparatus. The additional device may be an adjustable strap, band or belt configured to secure to the calf of the user.

In this case, the gait state recognizing apparatus may further include a wired or wireless communication device to transmit a result of measuring the movement by the IMU sensor 511.

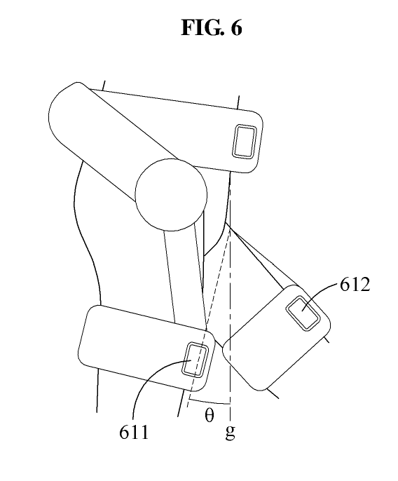

FIG. 6 illustrates an operation principle of a gait state recognizing apparatus according to at least one example embodiment.

Referring to FIG. 6, FIG. 6 illustrates a first inertial measurement unit (IMU) sensor 611 fastened to a thigh of a right leg to measure a movement of the right leg of a user and a second IMU sensor 612 fastened to a thigh of a left leg to measure a movement of the left leg of the user.

The first IMU sensor 611 and the second IMU sensor 612 may be provided to measure six degrees of freedom (6 DoF) movement of each leg. The gait state recognizing apparatus 100, 200 may calculate a rotation angle of each leg relative to a direction of gravity g based on the measured movement of each leg and calculate an angular velocity of each leg relative to the direction of gravity g based on a trend of the rotation angle of each leg relative to the direction of gravity g as time elapses.

For example, the gait state recognizing apparatus calculates a rotation angle Θ of the right leg relative to the direction of gravity g based on the movement of the right leg measured by the first IMU sensor 611. Here, a center of rotation associated with the rotation angle Θ may be a set (or, alternatively, preset) reference point. The reference point may be set to be a set (or, alternatively, predetermined) initial value or customized to be a point associated with an actual position of a hip joint of the user by the gait state recognizing apparatus.

In addition, the gait state recognizing apparatus 100, 200 may calculate the angular velocity of each leg relative to the direction of gravity g based on the trend of the rotation angle Θ of each leg as time elapses by continuously calculating the rotation angle Θ of each leg based on a result of continuously measuring the movement of each leg.

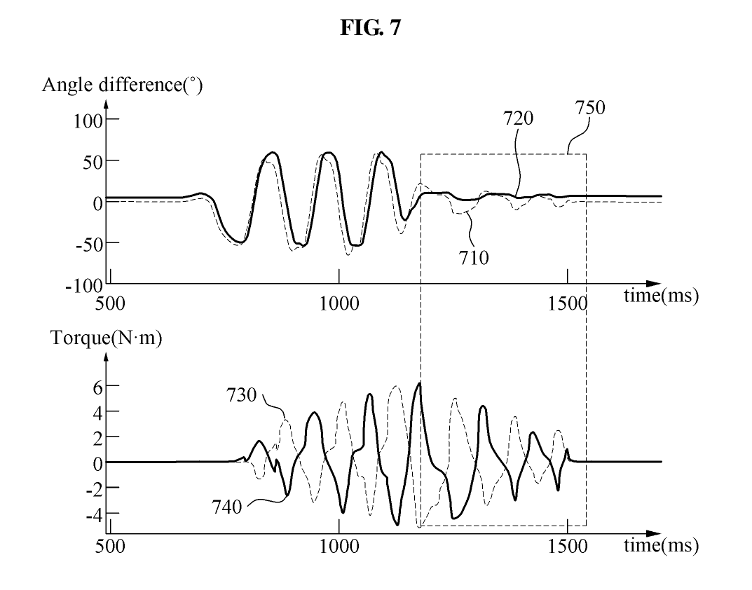

FIG. 7 is a graph illustrating an experimental result of a gait state recognizing apparatus according to at least one example embodiment.

Referring to FIG. 7, an upper graph of FIG. 7 represents a trend of a rotation angle 710 of a joint measured using an encoder sensor disposed around the joint of a user as time elapses and a trend of a rotation angle 720 of a joint measured using an inertial measurement unit (IMU) sensor disposed at a position where a rotation of the joint of the user has little influence as time elapses.

A lower graph of FIG. 7 represents a trend of a torque 730 to be applied to a right hip joint of the user and a trend of a torque 740 to be applied to a left hip joint of the user by a gait assistance apparatus while a rotation angle of a joint is measured using the encoder sensor or the IMU sensor, as time elapses.

A portion 750 which indicates the user has stopped walking may also indicate that a relative movement between the gait assistance apparatus and a body of the user is caused when the gait assistance apparatus applies a torque even though the user has stopped walking, such that a method of using the encoder sensor may falsely recognize the relative movement as a movement of the joint of the user. Compared to the method of using the encoder sensor, the method of using the IMU sensor may correctly recognize that the user has stopped walking.

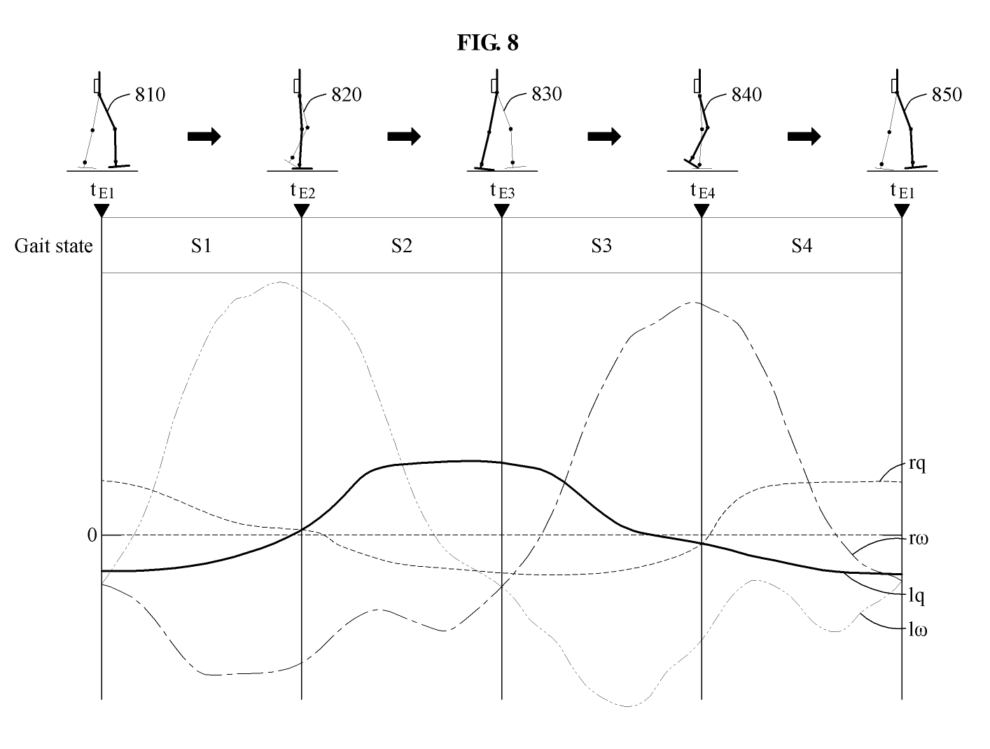

FIG. 8 is a graph illustrating a process of recognizing gait states during a gait cycle of a user using a gait state recognizing apparatus according to at least one example embodiment.

Referring to FIG. 8, the gait state recognizing apparatus 100, 200 may determine the gait state during a gait cycle of the user based on a trend of rotation angle rq and angular velocity r ω of a right leg of the user, and a trend of rotation angle lq and angular velocity l ω of a left leg of the user.

For example, a plurality of gait states included in the gait cycle of the user may include a gait state S1 in which a left leg swings, a gait state S2 in which the left leg lands, a gait state S3 in which a right leg swings, and a gait state S4 in which the right leg lands. The plurality of gait states may be identified based on a point in time at which the rotation angle q of one leg crosses the rotation angle of the other leg, and a point in time at which the angular velocity ω of one leg crosses the angular velocity of the other leg.

In a case of a general gait pattern in which the user walks in a forward direction, points in time tE1 and tE3 at which the angular velocity of one leg crosses the angular velocity of the other leg and points in time tE2 and tE4 at which the rotation angle of one leg crosses the rotation angle of the other leg may alternately appear.

For example, the point in time tE1 at which the angular velocity of each leg may appear in each of states 810 and 850 in which a distance between both legs is greatest in response to the user moving the right leg forward. Subsequently, the point in time tE2 at which the rotation angle of one leg crosses the rotation angle of the other leg may appear in a state 820 in which one leg crosses the other leg in response to the left leg of the user swinging. In addition, the point in time tE3 at which the angular velocity of one leg crosses the angular velocity of the other leg may appear in a state 830 in which the distance between both legs is greatest in response to the user moving the left leg forward. Subsequently, the point in time tE4 at which the rotation angle of one leg crosses the rotation angle of the other leg may appear in a state 840 in which one leg crosses the other leg in response to the right leg of the user swinging.

Thus, the gait state recognizing apparatus 100, 200 recognizes a transition between the gait states S1, S2, S3, and S4 during the gait cycle of the user based on the points in times tE1, tE2, tE3, and tE4. However, illustrated examples are provided only for description. The gait cycle and the gait states of the user may be defined differently from illustrated examples and are not limited to the illustrated examples.

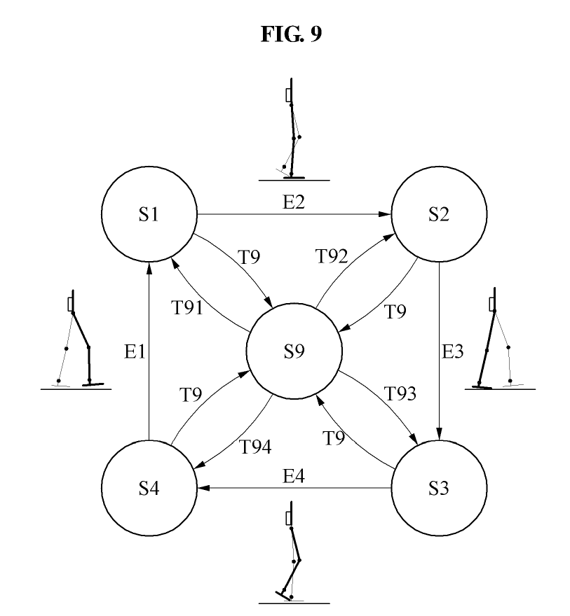

FIG. 9 illustrates a finite state machine (FSM) used to recognize a movement of a user by a gait state recognizing apparatus according to at least one example embodiment.

Referring to FIG. 9, the gait state recognizing apparatus 100, 200 may recognize a gait state of the user using the FSM including at least one state associated with a gait cycle of the user.

For example, the FSM may include a plurality of gait states defined as a gait state S1 in which a left leg swings, a gait state S2 in which the left leg lands, a gait state S3 in which a right leg swings, a gait state S4 in which the right leg lands, and an exceptional gait state S9. The gait state recognizing apparatus 100, 200 may use the FSM to determine whether transition conditions E1, E2, E3, and E4 between the gait states S1, S2, S3, and S4 are satisfied based on a trend of a rotation angle of each leg of the user and a trend of an angular velocity of each leg of the user.

For example, the transition conditions E1 and E3 may indicate whether the angular velocity w of one leg crosses the angular velocity w of the other leg, and the transition conditions E2 and E4 indicate whether the rotation angle q of one leg crosses the rotation angle q of the other leg. In addition, it may be determined that the transition condition T9, that is the condition of transition to the exceptional gait state S9 is satisfied when the gait state of the user cannot be recognized normally, and transition conditions T91, T92, T93, and T94, that is the conditions of transition to the respective gait states S1, S2, S3, and S4 are satisfied when the gait state of the user is recognizable as the exceptional gait state S9.

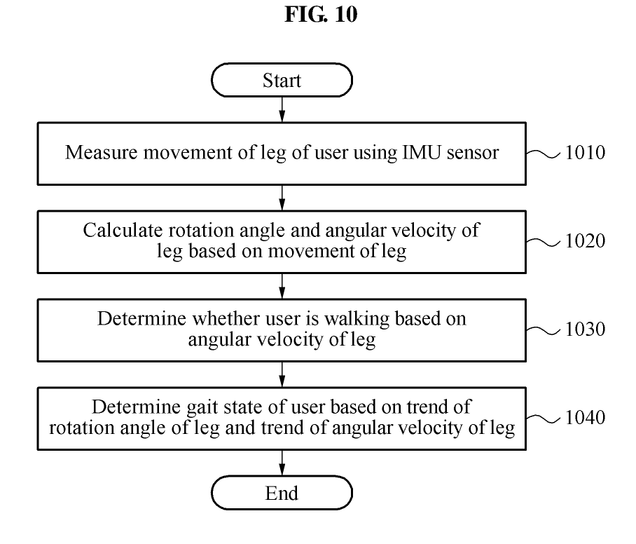

FIG. 10 is a flowchart illustrating a method of recognizing a gait state according to at least one example embodiment.

Referring to FIG. 10, in operation 1010, the gait state recognizing apparatus 100, 200 may measure a movement of at least one leg of a user using at least one inertial measurement unit (IMU) sensor. The IMU sensor may be disposed on a portion where a relative movement with respect to a body of a user is expected to have little influence. For example, the IMU sensor may be disposed on a surface of a thigh where a curvature is relatively low and a rotation of a joint is expected to have little influence.

In operation 1020, the gait state recognizing apparatus 100, 200 may calculate a rotation angle and an angular velocity of the at least one leg based on the movement of the at least one leg of the user. The gait state recognizing apparatus 100, 200 may calculate the rotation angle of the leg relative to a direction of gravity g based on the measured movement of the leg, and may calculate the angular velocity of the leg relative to the direction of gravity based on a trend of the calculated rotation angle.

In operation 1030, the gait state recognizing apparatus 100, 200 may determine whether the user is walking based on the angular velocity of the leg of the user. For example, the gait state recognizing apparatus 100, 200 may determine that the user is walking in response to an absolute value of the angular velocity being greater than a desired (or, alternatively, predetermined) threshold value. Through an operation of determining whether the user is walking, the gait state recognizing apparatus 100, 200 may recognize a gait state of the user only when the user is walking.

In operation 1040, the gait state recognizing apparatus 100, 200 may determine the gait state of the user based on a trend of the rotation angle of the leg of the user and a trend of the angular velocity of the leg of the user. For example, a plurality of gait states included in a gait cycle of the user may be defined in advance, and the gait state recognizing apparatus 100, 200 may determine the gait state of the user among the plurality of gait states based on the trend of the rotation angle and the trend of the angular velocity of the leg.

Thereby, by executing the method of recognizing the gait state, the gait state recognizing apparatus 100, 200 may obtain information associated with the gait state by measuring the movement of the leg relative to the direction of gravity using the IMU sensor without using an encoder sensor.

The units and/or modules described herein may be implemented using hardware components and software components. For example, the hardware components may include microphones, amplifiers, band-pass filters, audio to digital convertors, and processing devices. A processing device may be implemented using one or more hardware device configured to carry out and/or execute program code by performing arithmetical, logical, and input/output operations. The processing device(s) may include a processor, a controller and an arithmetic logic unit, a digital signal processor, a microcomputer, a field programmable array, a programmable logic unit, a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a processing device may include multiple processors or a processor and a controller. In addition, different processing configurations are possible, such a parallel processors.

The software may include a computer program, a piece of code, an instruction, or some combination thereof, to independently or collectively instruct and/or configure the processing device to operate as desired, thereby transforming the processing device into a special purpose processor. Software and data may be embodied permanently or temporarily in any type of machine, component, physical or virtual equipment, computer storage medium or device, or in a propagated signal wave capable of providing instructions or data to or being interpreted by the processing device. The software also may be distributed over network coupled computer systems so that the software is stored and executed in a distributed fashion. The software and data may be stored by one or more non-transitory computer readable recording mediums.

The methods according to the above-described example embodiments may be recorded in non-transitory computer-readable media including program instructions to implement various operations of the above-described example embodiments. The media may also include, alone or in combination with the program instructions, data files, data structures, and the like. The program instructions recorded on the media may be those specially designed and constructed for the purposes of example embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher level code that may be executed by the computer using an interpreter. The above-described devices may be configured to act as one or more software modules in order to perform the operations of the above-described example embodiments, or vice versa.

A number of example embodiments have been described above. Nevertheless, it should be understood that various modifications may be made to these example embodiments. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A gait state recognizing apparatus, comprising:
at least one inertial measurement unit (IMU) sensor configured to measure a movement of at least one leg of a user; and
a processor configured to,
calculate a rotation angle and an angular velocity of the at least one leg based on the measured movement of the at least one leg, and
determine a transition between gait states of the user based on one or more of the rotation angle and the angular velocity of the at least one leg.

2. The gait state recognizing apparatus of claim 1, wherein the at least one leg includes a left leg of the user and a right leg of the user, and
the processor is configured to,
determine the transition between the gait states based on one or more of (i) the rotation angle of the right leg crossing the rotation angle of the left leg and (ii) the angular velocity of the right leg crossing the angular velocity of the left leg,
calculate the rotation angle by calculating the rotation angle of the at least one leg relative to a direction of gravity from the movement of the at least one leg, and
calculate the angular velocity by calculating angular velocity of the at least one leg relative to the direction of gravity based on a trend of the rotation angle.

3. The gait state recognizing apparatus of claim 1, wherein the processor is configured to determine whether the user is walking based on the angular velocity of the at least one leg.

4. The gait state recognizing apparatus of claim 1, wherein the processor is configured to determine the gait states of the user based on a trend of the rotation angle and a trend of the angular velocity of the at least one leg.

5. The gait state recognizing apparatus of claim 4, wherein the processor is configured to determine the gait states using a finite state machine (FSM), the FSM including at least one state associated with a gait cycle of the user.

6. The gait state recognizing apparatus of claim 4, wherein the at least one leg includes a left leg of the user and a right leg of the user, and
the gait states include a state in which the left leg swings, a state in which the left leg lands, a state in which the right leg swings, and a state in which the right leg lands.

7. The gait state recognizing apparatus of claim 1, wherein the at least one leg includes a left leg of the user and a right leg of the user, and the at least one IMU sensor comprises:
a first IMU sensor configured to measure a movement of the right leg, and
a second IMU configured to measure a movement of the left leg.

8. The gait state recognizing apparatus of claim 7, wherein the at least one IMU sensor further comprises:
a third IMU sensor configured to measure a movement of an upper body of the user, and, wherein
the processor is configured to determine the gait states based on the movement of the upper body.

9. The gait state recognizing apparatus of claim 1, wherein one IMU sensor included in the at least IMU sensor is configured to measure movement associated with a portion of the user such that a relative movement of the portion with respect to the at least one leg is less than relative movement with respect to a joint of the at least one leg.

10. The gait state recognizing apparatus of claim 1, wherein one IMU sensor included in the at least one IMU sensor is configured to measure movement of a thigh of the user.

11. A method of recognizing a gate state, the method comprising:
measuring, via at least one inertial measurement unit (IMU) sensor, a movement of at least one leg of a user;
calculating a rotation angle and an angular velocity of the at least one leg based on the movement of the at least one leg;
determining whether the user is walking based on the angular velocity of the at least one leg; and
determining a transition between gait states of the user based on one or more of the rotation angle and the angular velocity of the at least one leg.

12. The method of claim 11, wherein
the at least one leg includes a left leg of the user and a right leg of the user,
the determining determines the transition between the gait states based on one or more of (i) the rotation angle of the right leg crossing the rotation angle of the left leg and (ii) the angular velocity of the right leg crossing the angular velocity of the left leg,
the calculating the rotation angle includes calculating the rotation angle of the at least one leg relative to a direction of gravity from the movement of the at least one leg, and
the calculating the angular velocity includes calculating the angular velocity of the at least one leg relative to the direction of gravity based on a trend of the rotation angle of the at least one leg.

13. The method of claim 11, further comprising:
determining the gait states of the user based on a trend of the rotation angle of the at least one leg and a trend of the angular velocity of the at least one leg when the user is walking.

14. The method of claim 11, wherein the determining the gait states determines the gait states based on a finite state machine (FSM), the FSM including at least one state associated with a gait cycle of the user.

15. The method of claim 11, wherein the at least one leg includes a left leg of the user and a right leg of the user, and at least one IMU sensor includes,
a first IMU sensor configured to measure a movement of the right leg and
a second IMU sensor configured to measure a movement of the left leg.

16. A non-transitory computer-readable storage medium storing a program that, when executed by a processor, configures the processor to perform the method of recognizing the gate state of claim 11.
